(19) United States
(12) Patent Application Publication
(10) Pub. No.: US 2024/0030491 A1
(43) Pub. Date: Jan. 25, 2024

(54) COMPOSITION

(71) Applicant: Mexichem Fluor S.A. de C.V., San Luis Potosi (MX)

(72) Inventors: Andrew SHARRATT, Runcorn (GB); Miodrag OLJACA, Concord, MA (US); Ira SAXENA, Runcorn (GB); John MCCARTHY, Runcorn (GB)

(21) Appl. No.: 17/767,635

(22) PCT Filed: Oct. 9, 2020
(86) PCT No.: PCT/GB2020/052526

§ 371 Date: Apr. 8, 2022

Related U.S. Application Data
(60) Provisional application No. 62/913,244, filed on Oct. 10, 2019.

(30) Foreign Application Priority Data
Nov. 11, 2019 (GB) 1916374.0
May 6, 2020 (GB) 2006716.1

(51) Int. Cl. H01M 10/0568; H01M 10/0567; H01M 10/0569

(52) U.S. Cl. CPC H01M 10/0568; H01M 10/0567; H01M 10/0569; H01M 2300/0037

(57) ABSTRACT

Use of a compound of Formula (1) in a nonaqueous battery electrolyte formulation: (Formula (1)) wherein R is H, F, Cl, CF₃, alkylr fluoroalkyl.

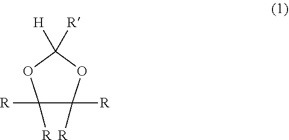

The present disclosure relates to nonaqueous electrolytic solutions for energy storage devices including batteries and capacitors, especially for secondary batteries and devices known as supercapacitors.

There are two main types of batteries: primary and secondary. Primary batteries are also known as non-rechargeable batteries. Secondary batteries are also known as rechargeable batteries. A well-known type of rechargeable battery is the lithium-ion battery. Lithium-ion batteries have a high energy density, no memory effect and low self-discharge.

Lithium-ion batteries are commonly used for portable electronics and electric vehicles. In the batteries lithium ions move from the negative electrode to the positive electrode during discharge and back when charging.

Typically, the electrolytic solutions include a nonaqueous solvent and an electrolyte salt, plus additives. The electrolyte is typically a mixture of organic carbonates such as ethylene carbonate, propylene carbonate, fluoroethylene carbonate, dialkyl carbonates such as ethyl methyl carbonate and ethers and polyethers such as dimethoxyethane containing a lithium-ion electrolyte salt. Many lithium salts can be used as the electrolyte salt; common examples include lithium hexafluorophosphate (LiPF₆), lithium bis (fluorosulfonyl) imide (LiFSI) and lithium bis(trifluoromethanesulfonyl)imide (LiTFSI).

The electrolytic solution has to perform a number of separate roles within the battery.

The principal role of the electrolyte is to facilitate the flow of charge carriers between the cathode and anode. This occurs by transportation of metal ions within the battery to or from one or both of the anode and cathode, whereby on chemical reduction or oxidation, electrical charge is liberated/adopted.

Thus, the electrolyte needs to provide a medium which is capable of solvating and/or supporting the metal ions.

Due to the use of lithium electrolyte salts and the interchange of lithium ions with lithium metal, which is very reactive with water, as well as the sensitivity of other battery components to water, the electrolyte is usually non-aqueous.

Additionally, the electrolyte has to have suitable rheological properties to permit/enhance the flow of ions therein, at the typical operating temperature to which a battery is exposed and is expected to perform.

Moreover, the electrolyte has to be as chemically inert as possible. This is particularly relevant in the context of the expected lifetime of the battery regarding internal corrosion within the battery (e.g. of the electrodes and casing) and the issue of battery leakage. Also of importance within the consideration of chemical stability is flammability. Unfortunately, typical electrolyte solvents can be a safety hazard, since they often comprise a flammable material.

This can be problematic as in operation, when discharging or being discharged, batteries may accumulate heat. This is especially true for high density batteries such as lithium-ion batteries. It is therefore desirable that the electrolyte displays a low flammability, with other related properties such as a high flash point.

It is also desirable that the electrolyte does not present an environmental issue with regard to disposability after use or other environmental issue such as global warming potential.

“Regioselectivity in addition reactions of some binucleophilic reagents to (trifluoromethyl) acetylene” Stepanova et. al., Zhurnal Organicheskoi Khimii (1988), 24(4), 692-9 describes the preparation of a dioxolane having a CF₃CH₂ group, at relatively low levels of selectivity.

The listing or discussion of an independently prior published document in this specification should not necessarily be taken as an acknowledgement that the document is part of the state of the art or is common general knowledge.

It is an object of the present invention to provide a nonaqueous electrolytic solution, which provides improved properties over the nonaqueous electrolytic solution of the prior art.

It is a further object of the invention to provide an improved method of manufacturing the dioxolanes used according to the invention.

Use Aspects

According to a first aspect of the invention there is provided the use of a compound of Formula 1 in a nonaqueous battery electrolyte formulation.

According to a second aspect of the invention there is provided the use of a nonaqueous battery electrolyte formulation comprising a compound of Formula 1 in a battery.

Composition/Device Aspects

According to a third aspect of the invention there is provided a battery electrolyte formulation comprising a compound of Formula 1.

According to a fourth aspect of the invention there is provided a formulation comprising a metal ion and a compound of Formula 1, optionally in combination with a solvent.

According to a fifth aspect of the invention there is provided a battery comprising a battery electrolyte formulation comprising a compound of Formula 1.

Method Aspects

According to a sixth aspect of the invention there is provided a method of reducing the flash point of a battery and/or a battery electrolyte formulation, comprising the addition of a formulation comprising a compound of Formula 1.

According to a seventh aspect of the invention there is provided a method of powering an article comprising the use of a battery comprising a battery electrolyte formulation comprising a compound of Formula 1.

According to an eighth aspect of the invention there is provided a method of retrofitting a battery electrolyte formulation comprising either (a) at least partial replacement of the battery electrolyte with a battery electrolyte formulation comprising a compound of Formula 1, and/or (b) supplementation of the battery electrolyte with a battery electrolyte formulation comprising a compound of Formula 1.

According to a ninth aspect of the invention there is provided a method of preparing a battery electrolyte formulation comprising mixing a compound of Formula 1 with a lithium containing salt and other solvents or co-solvents.

According to a tenth aspect of the invention there is provided a method of preparing a battery electrolyte formulation comprising mixing a composition comprising a compound of Formula 1 with a lithium-containing compound.

According to an eleventh aspect of the invention there is provided a method of improving battery capacity/charge transfer within a battery/battery life/etc. by the use of a compound of Formula 1.

According to a twelfth aspect of the invention there is provided a method of reducing the overpotential generated at one or both of the electrodes of a battery during cycling by the use of a compound of Formula 1.

Process Aspects

According to a thirteenth aspect, there is provided a method of manufacturing a compound of Formula 1

Compound of Formula 1

In reference to all aspects of the invention the preferred embodiment of Formula (1) is below:

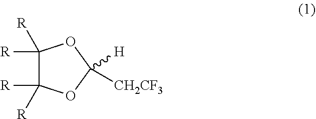

wherein R=H, F, CF₃, alkyl or fluoroalkyl.

“Regioselectivity in addition reactions of some binucleophilic reagents to (trifluoromethyl) acetylene” Stepanova et. al., Zhurnal Organicheskoi Khimii (1988), 24(4), 692-9 describes the preparation of a dioxolane of Formula 1 with all 4 Rs=H, as a minor by-product in the reaction of a compound of Formula RNH₂ with R=ethyl or phenyl and trifluoromethyl acetylene with base, with a solvent of ethylene glycol. The trifluoromethyl acetylene is condensed in the potassium hydroxide/methylene glycol solution at −70° C., which is then warmed to room temperature and thereafter to 80° C. for 5 hours. The reaction products are said to include the linear adduct 2-(3,3,3-trifluoro-1Z-propenyloxy) ethanol and the cyclic adduct 2-(2,2,2-trifluoroethyl)-1,3-dioxolane, at a ratio of 4:1.

Suitable dioxalanes can also be produced by the methods demonstrated in this application.

We have surprisingly found that a high yield of the cyclic adduct can be attained in this reaction if the trifluoromethyl acetylene (TFMA) component is maintained at a positive pressure in the reaction vessel.

Hence in a further aspect of the invention there is provided a method of manufacturing a compound of Formula 1 by reacting a glycol with trifluoromethyl acetylene at positive pressure under basic conditions.

Preferably, the reaction is carried out at 0° C. or above, conveniently at 20° C. or above, conveniently at a temperature of around 40° C. Preferably, the base is KOH.

Conveniently, the glycol is reacted with the TFMA for a period of at least one hour, preferably at least five hours and preferably at least 9 to 10 hours. Ideally the reaction time should be less than five days. We have found that a convenient reaction time is approximately 72 hours.

The pressure during the reaction is preferably at least 2 barg, preferably at least 4 barg, preferably at least 6 barg. We have found that a convenient pressure for the reaction is between 8 and 12 barg, preferably around 10 barg. In a preferred embodiment, the gas pressure is monitored and maintained during the reaction, if necessary topping-up the reaction vessel with TFMA during the reaction.

We have further surprisingly found that a high yield of the cyclic adduct can be attained if a diol or glycol is condensed with an aldehyde:

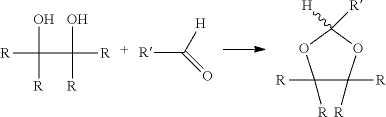

Hence in a further aspect of the invention there is provided a method of manufacturing a compound of Formula 1 by reacting a glycol or diol with an aldehyde.

Preferably, the diol or glycol is a compound of Formula 2a:

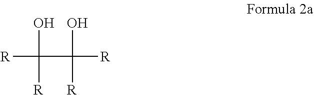

In the compound of formula 2a each R group can independently comprise of functional groups that include H, F, Cl, CF₃, alkyl, fluoroalkyl etc.

Preferably, the aldehyde is a compound of Formula 2b:

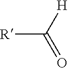

In the compound of Formula 1 and Formula 2b R′ can comprise of functional groups that include F, Cl, CF₃, alkyl, fluoroalkyl etc. Conveniently R′ is the same as R. In a preferred embodiment, R′ is CH₂CF₃; also in a preferred embodiment, R is H and/or CF₃. Conveniently. R′ is CH₂CFS and R is H and/or CF₃.

The table below includes some examples of preferred diols, aldehydes and the products of their condensation reactions:

| Diol | Aldehyde | Product |
|---|---|---|
| HOCH₂CH₂OH | CF₃CHO | 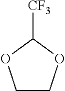 |
| HOCH₂CH₂OH | CF₃CH₂CHO | 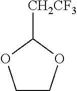 |
| CF₃CHOHCH₂OH | CF₃CHO | 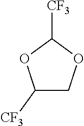 [SMILES: FC(F)(F)C1COC(C(F)(F)F)O1] |
| CF₃CH(OH)CH₂OH | CF₃CH₂CHO | 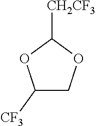 |
| CF₃CH(OH)CH(OH)CF3 | CF₃CHO | 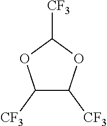 [SMILES: FC(F)(F)C1OC(C(F)(F)F)C(C(F)(F)F)O1] |
| CF₃CH(OH)CH(OH)CF3 | CF₃CH₂CHO | 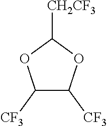 |

The products of these reactions includes, all stereoisomers some of which may possess different properties e.g. melting point, boiling point or electrochemical.

Conveniently, the glycol or diol is reacted with the aldehyde for a period of at least twelve hours, preferably at least twenty-four hours and preferably at least 48 hours. Ideally the reaction time should be less than five days. We have found that a convenient reaction time is approximately 48 hours.

The yield of the reaction can be improved by continuously removing the water by-product as it is formed. The reaction can be conducted at any suitable temperature and pressure such that the water by-product can be efficiently removed. Alternatively, the reaction can be conducted in the presence of an agent that removes the water as it is formed e.g. a molecular sieve or zeolite, sulphuric acid or thionyl chloride.

The diol and aldehyde can be present in equal amounts or an excess of one over the other can be used. A reaction solvent can be advantageously used to ensure good contacting between diol and aldehyde. An example of a suitable reaction solvent is dichloromethane.

A catalyst can be used to increase the rate of reaction and improve yields and selectivity. Preferably the catalyst is an acid, such as for example p-toluene sulphonic acid.

For use in battery electrolyte compositions, it is essential that preparative procedures are high yielding and selective such that it is possible to recover the compound of Formula 1 and purify it to greater than 95%, for example greater than 99%.

Thus, another objective of this application to improve on the known methods for preparing compounds of Formula 1, recovering them and purifying them to greater than 95%, for example greater than 99% purity. Compounds of Formula 1 can be conveniently prepared in high yield and selectively by reaction of TFMA with a glycol compound, preferably of Formula 2 and under basic conditions, with heating at pressure, where the pressure inside the reactor is maintained by repeatedly dosing it with TFMA:

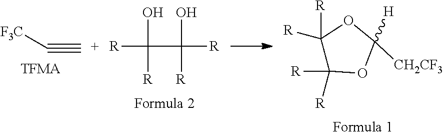

In an embodiment, the alkyl or fluoroalkyl group may have a carbon chain length of C₁-C₆.

Preferably, by “alkyl” is meant C₁-C₆. By “fluoroalkyl” is meant an alkyl group that is partially- or fully-fluorinated.

In a preferred embodiment, at least one of the R groups can be CF₃. Conveniently, one, two, three or four R groups can be CF₃.

Compounds of Formula 1 can also be conveniently prepared in high yield and selectively by reaction of an aldehyde with a glycol compound, preferably of Formula 2a and under acidic and dehydrating conditions:

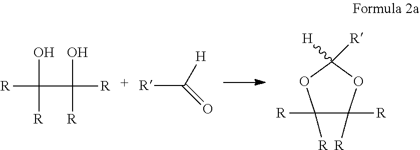

In an embodiment, the alkyl or fluoroalkyl group may have a carbon chain length of C₁-C₆.

Preferably, by “alkyl” is meant C₁-C₆. By “fluoroalkyl” is meant an alkyl group that is partially- or fully-fluorinated.

In a preferred embodiment, at least one of the R groups can be CF₃. Conveniently, one, two, three or four R groups can be CF₃.

### Advantages

In the aspects of the invention, the electrolyte formulation has been found to be surprisingly advantageous.

The advantages of using compounds of Formula 1 in electrolyte solvent compositions manifest themselves in a number of ways. Their presence can reduce the flammability of the electrolyte composition (such as when for example measured by flashpoint). Their oxidative stability makes them useful for batteries required to work in harsh conditions and at high temperatures, they are compatible with common electrode chemistries and can even enhance the performance of these electrodes through their interactions with them.

Additionally, electrolyte compositions comprising compounds of Formula 1 may have superior physical properties including low viscosity and a low melting point, yet a high boiling point with the associated advantage of little or no gas generation in use. The electrolyte formulation may wet and spread extremely well over surfaces, particularly fluorine-containing surfaces; this is postulated to result from a beneficial a relationship between its adhesive and cohesive forces, to yield a low contact angle.

Furthermore, electrolyte compositions that comprise compounds of Formula 1 may have superior electro-chemical properties, including improved capacity retention, reduced overpotential generation at one or both electrodes during cycling, improved cyclability and capacity retention, improved compatibility with other battery components e.g. separators and current collectors, and with all types of cathode and anode chemistries, including systems that operate across a range of voltages and especially high voltages, and which include additives such as silicon. In addition, the electrolyte formulations display good solvation of metal (e.g. lithium) salts and interaction with any other electrolyte solvents present.

Preferred features relating to the aspects of the invention follows below. Preferences and options for a given aspect, feature or parameter of the invention should, unless the context indicates otherwise, be regarded as having been disclosed in combination with any and all preferences and options for all aspects, features and parameters of the invention.

Preferred Compounds

Compound of Formula 1 has the structure:

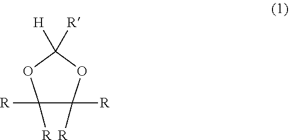

wherein R and R′=H, F, Cl, CF₃, alkyl or fluoroalkyl.

In a further preferred embodiment, at least one of the H groups can be replaced with CF₃ groups for example one, two or three H groups can be CF₃.

Preferably the compound of Formula 1 is prepared by a method that facilitates it recovery and purification to greater than 95%, for example greater than 99%.

Preferably compound of Formula 1 has the structure:

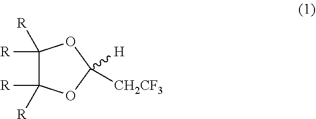

wherein R=H, F, CF₃, alkyl or fluoroalkyl.

In a preferred embodiment compound of Formula 1 is:

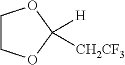

In a further preferred embodiment, at least one of the R groups can be CF₃. Conveniently, one, two, three or four R groups can be CF₃.

Preferably the compound of Formula 1 is prepared by a method that facilitates it recovery and purification to greater than 95%, for example greater than 99%.

Electrolyte Formulation

The electrolyte formulation will preferably comprise 0.1 wt % to 99.9 wt % of the compound of Formula 1, conveniently 90.0 wt % to 99.9 wt % of the compound of Formula 1.

Metal Salts

The nonaqueous electrolytic solution further comprises a metal electrolyte salt, typically present in an amount of 0.1 to 20 wt % relative to the total mass of the nonaqueous electrolyte formulation.

The metal salt generally comprises a salt of lithium, sodium, magnesium, calcium, lead, zinc or nickel.

Preferably the metal salt comprises a salt of lithium, such as those selected from the group comprising lithium hexafluorophosphate (LiPF₆), lithium hexafluoroarsenate monohydrate (LiAsF₆), lithium perchlorate (LiClO₄), lithium tetrafluoroborate (LiBF₄), lithium triflate (LiSO₃CF₃), lithium bis(fluorosulfonyl)imide (LiFSI, Li(FSO₂)₂N) and lithium bis(trifluoromethanesulfonyl)imide (LiTFSI, Li(CF₃SO₂)₂N).

Most preferably, the metal salt comprises LiPF₆, LiFSI or LiTFSI. Thus, in a most preferred variant of the fourth aspect of the invention, there is provided a formulation comprising LiPF₆, LiFSI, LITFSI and a compound of Formula 1, optionally in combination with one or more co-solvents.

Solvents

The nonaqueous electrolytic solution may comprise a solvent. Preferred examples of solvents include fluoroethylene carbonate (FEC) and/or propylene carbonate (PC), dimethyl carbonate (DMC), ethyl methyl carbonate (EMC), ethylene carbonate (EC) or dimethoxyethane (DME).

Where present, the additional solvent makes up from 0.1 wt % to 99.9 wt % of the liquid component of the electrolyte.

Additives

The nonaqueous electrolytic solution may include an additive.

Suitable additives may serve as surface film-forming agents, which form an ion permeable film on the surface of the positive electrode or the negative electrode. This can pre-empt a decomposition reaction of the nonaqueous electrolytic solution and the electrolyte salt occurring on the surface of the electrodes, thereby preventing the decomposition reaction of the nonaqueous electrolytic solution on the surface of the electrodes.

Examples of film-forming agent additives include vinylene carbonate (VC), ethylene sulfite (ES), lithium bis(oxalato)borate (LiBOB), cyclohexylbenzene (CHB) and ortho-terphenyl (OTP). The additives may be used singly, or two or more may be used in combination.

When present, the additive is present in an amount of 0.1 to 3 wt % relative to the total mass of the nonaqueous electrolyte formulation.

Battery

Primary/Secondary Battery

The battery may comprise a primary (non-rechargeable) or a secondary battery (rechargeable). Most preferably the battery comprises a secondary battery.

A battery comprising the nonaqueous electrolytic solutions will generally comprise several elements. Elements making up the preferred nonaqueous electrolyte secondary battery cell are described below. It is appreciated that other battery elements may be present (such as a temperature sensor); the list of battery components below is not intended to be exhaustive.

Electrodes

The battery generally comprises a positive and a negative electrode. Usually the electrodes are porous and permit metal ions (lithium ions) to move in and out of their structures with a process called insertion (intercalation) or extraction (deintercalation).

For rechargeable batteries (secondary batteries), the term cathode designates the electrode where reduction is taking place during the discharge cycle. For lithium-ion cells the positive electrode (“cathode”) is the lithium-based one.

Positive Electrode (Cathode)

The positive electrode is generally composed of a positive electrode current collector such as a metal foil, optionally with a positive electrode active material layer disposed on the positive electrode current collector.

The positive electrode current collector may be a foil of a metal that is stable at a range of potentials applied to the positive electrode, or a film having a skin layer of a metal that is stable at a range of potentials applied to the positive electrode. Aluminium (AI) is desirable as the metal that is stable at a range of potentials applied to the positive electrode.

The positive electrode active material layer generally includes a positive electrode active material, and other components such as a conductive agent and a binder. This is generally obtained by mixing the components in a solvent, applying the mixture onto the positive electrode current collector, followed by drying and rolling.

The positive electrode active material may be lithium (Li) or a lithium-containing transition metal oxide. The transition metal element may be at least one selected from the group consisting of scandium (Sc), manganese (Mn), iron (Fe), cobalt (Co), nickel (Ni), copper (Cu) and yttrium (Y). Of these transition metal elements, manganese, cobalt and nickel are the most preferred.

Further, in certain embodiments transition metal fluorides may be preferred.

Some of the transition metal atoms in the transition metal oxide may be replaced by atoms of a non-transition metal element. The non-transition element may be selected from the group consisting of magnesium (Mg), aluminium (Al), lead (Pb), antimony (Sb) and boron (B). Of these non-transition metal elements, magnesium and aluminium are the most preferred.

Preferred examples of positive electrode active materials include lithium-containing transition metal oxides such as LiCoO₂, LiNiO₂, LiMn₂O₄, LiMnO₂, LiNi_{1-y}Co_{y}O₂ (0<y<1), LiNi_{1-y-z}Co_{y}Mn_{z}O₂ (0<y+z<1) and LiNi_{1-y-z}Co_{y}Al_{z}O₂ (0<y+z<1). LiNi_{1-y-z}Co_{y}Mn_{z}O₂ (0<y+z<0.5) and LiNi_{1-y-z}Co_{y}Al_{z}O₂ (0<y+z<0.5) containing nickel in a proportion of not less than 50 mol % relative to all the transition metals are desirable from the perspective of cost and specific capacity. These positive electrode active materials contain a large amount of alkali components and thus accelerate the decomposition of nonaqueous electrolytic solutions to cause a decrease in durability. However, the nonaqueous electrolytic solution of the present disclosure is resistant to decomposition even when used in combination with these positive electrode active materials.

The positive electrode active material may be a lithium (Li) containing transition metal fluoride. The transition metal element may be at least one selected from the group consisting of scandium (Sc), manganese (Mn), iron (Fe), cobalt (Co), nickel (Ni), copper (Cu) and yttrium (Y). Of these transition metal elements, manganese, cobalt and nickel are the most preferred.

Some of the transition metal atoms in the transition metal fluoride may be replaced by atoms of a non-transition metal element. The non-transition element may be selected from the group consisting of magnesium (Mg), aluminium (AI), lead (Pb), antimony (Sb) and boron (B). Of these non-transition metal elements, magnesium and aluminium are the most preferred.

A conductive agent may be used to increase the electron conductivity of the positive electrode active material layer. Preferred examples of the conductive agents include conductive carbon materials, metal powders and organic materials. Specific examples include carbon materials as acetylene black, ketjen black and graphite, metal powders as aluminium powder, and organic materials as phenylene derivatives.

A binder may be used to ensure good contact between the positive electrode active material and the conductive agent, and to increase the adhesion of the components such as the positive electrode active material with respect to the surface of the positive electrode current collector. Preferred examples of the binders include fluoropolymers and rubber polymers, such as polytetrafluoroethylene (PTFE), polyvinylidene fluoride (PVdF) ethylene-propylene-isoprene copolymer and ethylene-propylene-butadiene copolymer. The binder may be used in combination with a thickener such as carboxymethylcellulose (CMC) or polyethylene oxide (PEO).

Negative Electrode (Anode)

The negative electrode is generally composed of a negative electrode current collector such as a metal foil, optionally with a negative electrode active material layer disposed on the negative electrode current collector.

The negative electrode current collector may be a foil of a metal. Copper (lithium-free) is suitable as the metal. Copper is easily processed at low cost and has good electron conductivity.

Generally, the negative electrode comprises carbon, such as graphite or graphene or lithium metal. In a preferred embodiment, the negative electrode is lithium metal.

Silicon based materials can also be used for the negative electrode. A preferred form of silicon is in the form of nano-wires, which are preferably present on a support material. The support material may comprise a metal (such as steel) or a non-metal such as carbon.

The negative electrode may include an active material layer. Where present the active material layer includes a negative electrode active material and other components such as a binder. This is generally obtained by mixing the components in a solvent, applying the mixture onto the positive electrode current collector, followed by drying and rolling.

Negative electrode active materials are not particularly limited, provided the materials can store and release lithium ions. Examples of suitable negative electrode active materials include carbon materials, metals, alloys, metal oxides, metal nitrides, and lithium-intercalated carbon and silicon. Examples of carbon materials include natural/artificial graphite, and pitch-based carbon fibres. Preferred examples of metals include lithium (Li), silicon (Si), tin (Sn), germanium (Ge), indium (In), gallium (Ga), titanium (Ti), lithium alloys, silicon alloys and tin alloys. Examples of lithium-based material include lithium titanate (Li₂TiO₃).

The active material may can be in many forms such as a thin film, foil or supported on a three-dimensional matrix.

As with the positive electrode, the binder may be a fluoropolymer or a rubber polymer and is desirably a rubbery polymer, such as styrene-butadiene copolymer (SBR). The binder may be used in combination with a thickener.

In a preferred embodiment, the negative electrode is lithium metal, in a secondary battery; conveniently in such embodiments, but also in other embodiments with other negative electrodes and in other battery types, the electrolyte comprises LiTFSI and/or LiFSI, dimethoxyethane, and a compound of Formula 1.

Separator

A separator is preferably present between the positive electrode and the negative electrode. The separator has insulating properties. The separator may comprise a porous film having ion permeability. Examples of porous films include microporous thin films, woven fabrics and nonwoven fabrics. Suitable materials for the separators are polyolefins, such as polyethylene and polypropylene.

Case

The battery components are preferably disposed within a protective case.

The case may comprise any suitable material which is resilient to provide support to the battery and an electrical contact to the device being powered.

In one embodiment the case comprises a metal material, preferably in sheet form, moulded into a battery shape. The metal material preferably comprises a number of portions adaptable be fitted together (e.g. by push-fitting) in the assembly of the battery. Preferably the case comprises an iron/steel-based material.

In another embodiment the case comprises a plastics material, moulded into a battery shape. The plastics material preferably comprises a number of portions adaptable be joined together (e.g. by push-fitting/adhesion) in the assembly of the battery. Preferably the case comprises a polymer such as polystyrene, polyethylene, polyvinyl chloride, polyvinylidene chloride, or polymonochlorofluoroethylene. The case may also comprise other additives for the plastics material, such as fillers or plasticisers. In this embodiment wherein the case for the battery predominantly comprises a plastics material, a portion of the casing may additionally comprise a conductive/metallic material to establish electrical contact with the device being powered by the battery.

Arrangement

The positive electrode and negative electrode may be wound or stacked together through a separator. Together with the nonaqueous electrolytic solution they are accommodated in the exterior case. The positive and negative electrodes are electrically connected to the exterior case in separate portions thereof.

Module/Pack

A number/plurality of battery cells may be made up into a battery module. In a battery module the battery cells may be organised in series and/or in parallel. Typically, these are encased in a mechanical structure.

A battery pack may be assembled by connecting multiple modules together in a series or parallel. Typically, battery packs include further features such as sensors and controllers including battery management systems and thermal management systems. The battery pack generally includes an encasing housing structure to make up the final battery pack product.

End Uses

The battery of the invention, in the form of an individual battery/cell, module and/or pack (and the electrolyte formulations therefor) are intended to be used in one or more of a variety of end products.

Preferred examples of end products include portable electronic devices, such as GPS navigation devices, cameras laptops, tablets and mobile phones. Other preferred examples of end products include vehicular devices (as provision of power for the propulsion system and/or for any electrical system or devices present therein), such as electrical bicycles and motorbikes, as well as automotive applications (including hybrid and purely electric vehicles).

Preferences and options for a given aspect, feature or parameter of the invention should, unless the context indicates otherwise, be regarded as having been disclosed in combination with any and all preferences and options for all other aspects, features and parameters of the invention.

The invention will now be illustrated with reference to the following non-limiting examples.

### EXAMPLES

### Example 1—Synthesis, Isolation and Electrochemical Testing of 2-(2,2,2-trifluoroethyl)-1,3-Dioxolane (Mexi-20)

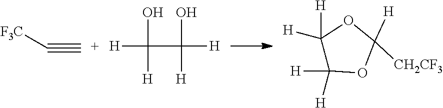

Potassium hydroxide (4.02 g 85% wt) was dissolved in ethylene glycol (20 g) with stirring in a 100 ml pressure reactor vessel. Once dissolution was complete the reactor vessel was sealed, purged with nitrogen and the contents heated to 40° C. with stirring before being pressurised with trifluoromethyl acetylene (TFMA) to 8 barg. After 52 minutes the pressure had dropped to 6.4 barg and was re-pressurised to 10 barg with more TFMA. This pattern of pressure loss and re-pressurisation was repeated several times over 6 hours before the final pressurisation to 10 barg with TFMA. After 72 hours further stirring at 40° C. the final pressure in the reactor vessel was 6.4 barg.

After cooling and depressurisation, the contents of the reactor were recovered as a viscous yellow oil. To this oil was added 21 g of water which affected a phase separation. The lower organic layer was recovered and repeatedly washed with 50 ml aliquots of water. The product was dried over anhydrous sodium sulphate to yield 16.1 g of product.

The crude product was analysed by GC-MS which showed that it comprised the desired product and an unsaturated ether by-product identified as CF₃CH═CHOCH₂CH₂OH in the ratio 6.1:1.

The desired product was separated from the by-products in the crude product by distillation and was analysed by ¹⁹F NMR (56 MHz) δ −64.5 (t, J=11.0 Hz). The mass spectrum of the desired product contained characteristic fragments at m/z 155, 126, 111, 73, 69, 45.

For preparative purposes this procedure was scaled-up. Thus, KOH (40 g. 85%) was dissolved in ethylene glycol (200 g) and transferred to a 450 ml Hastelloy autoclave. The autoclave was sealed, pressure tested and purged with nitrogen before the contents were heated to 40° C. with stirring. When at temperature the autoclave was pressurised with TFMA to 9-10 Barg. The pressure inside the vessel dropped as the TFMA reacted. When the pressure had dropped to around 3 barg the vessel was re-pressurised with TFMA. This cycle of reaction and re-pressurisation steps was continued until the rate of TFMA consumption became negligible. In a typical procedure the reactor would be re-pressurised 5-6 times over the course of 3 days or so.

Five batches of crude product were prepared. The crude product was separated from the reaction mixtures by quenching with water, which caused the product to separate allowing it to be recovered. The separated crude product was further washed with water to remove traces of potassium salts before being dried over sodium sulphate. The product from each of the batches were combined to yield 269 g of a pale-yellow oil which was analysed by GCMS and found to comprise 93% of the desired product 2-(2,2,2-trifluoroethyl)-1,3-dioxolane.

The crude product was further purified by distillation using a packed column which yielded the following fractions:

| | Boiling point | Mass | Purity |
|---|---|---|---|
| Fraction | (° C.) | (g) | (GCMS, area %) |
| 1 | 110 | 44.0 | 92.5 |
| 2 | 114 | 36.5 | 98.9 |
| 3 | 114 | 50.4 | 99.5 |
| 4 | 114 | 43.5 | 99.5 |
| 5 | 114 | 9.3 | 99.1 |
| 6 | 114 | 6.3 | 99.1 |
| Residue | — | Balance | 87.7 |

Fractions 2-6 were combined and analysed by a combination of GC-MS and multi-nuclear NMR spectroscopy:

Purity (GC-MS Area %) 99.1

Mass spectrum m/z: 155, 126, 111, 91, 77, 73, 69, 57, 45, 43

¹H NMR (60 MHz) δ 5.12 (t, J=5.12 Hz, 1H), 3.92 (d, J=2.1 Hz, 4H), 2.48 (qd, J=10.9, 4.8 Hz, 2H)

¹⁹F NMR (56 MHz) δ −64.5 (t, J=11.0 Hz)

¹³C NMR (15 MHz) δ 125.97 (q, j=276.2 Hz), 99.37 (q, 3.9 Hz), 65.46 (s), 39.47 (q, 27.6 Hz)

This spectral information unequivocally confirms that the purified product was the desired compound of Formula 1: 2-(2,2,2-trifluoroethyl)-1,3-Dioxolane (Mexi-20). Furthermore, the data serves to demonstrate that it can be made in high yield and selectively such that it can be purified to greater than 99% and so be of utility in lithium ion and lithium metal battery electrolyte compositions.

Compositions of the Invention (all % w/w):

TABLE 1
Compositions comprising 2-(2,2,2-trifluoroethyl)-1,3-
Dioxolane and lithium hexafluorophosphate (LiPF₆)

| Base composition | Additive | FIG. |
|---|---|---|
| 95% 1M LiPF₆ in Propylene | 5% 2-(2,2,2-trifluoroethyl)- | 1a |
| carbonate | 1,3-Dioxolane | |
| 85% 1M LiPF₆ in Propylene | 15% 2-(2,2,2-trifluoroethyl)- | 1b |
| carbonate | 1,3-Dioxolane | |
| 25% 1M LiPF₆ in Propylene | 75% 2-(2,2,2-trifluoroethyl)- | 1c |
| carbonate | 1,3-Dioxolane | |
| 95% 1M LiPF₆ in a mixture of | 5% 2-(2,2,2-trifluoroethyl)- | 2a |
| Propylene carbonate (90%) and | 1,3-Dioxolane | |
| fluoroethylene carbonate (10%) | | |
| 85% 1M LiPF₆ in a mixture of | 15% 2-(2,2,2-trifluoroethyl)- | 2b |
| Propylene carbonate (90%) and | 1,3-Dioxolane | |
| fluoroethylene carbonate (10%) | | |
| 25% 1M LiPF₆ in a mixture of | 75% 2-(2,2,2-trifluoroethyl)- | 2c |
| Propylene carbonate (90%) and | 1,3-Dioxolane | |
| fluoroethylene carbonate (10%) | | |
| 95% 1M LiPF₆ in a mixture of | 5% 2-(2,2,2-trifluoroethyl)- | 3a |
| ethylene carbonate (30%) and | 1,3-Dioxolane | |
| ethyl methyl carbonate (70%) | | |
| 85% 1M LiPF₆ in a mixture of | 15% 2-(2,2,2-trifluoroethyl)- | 3b |
| ethylene carbonate (30%) and | 1,3-Dioxolane | |
| ethyl methyl carbonate (70%) | | |
| 25% 1M LiPF₆ in a mixture of | 75% 2-(2,2,2-trifluoroethyl)- | 3c |
| ethylene carbonate (30%) and | 1,3-Dioxolane | |
| ethyl methyl carbonate (70%) | | |

TABLE 2
Compositions comprising 2-(2,2,2-trifluoroethyl)-1,3-Dioxolane
and lithium bis(fluorosulfonyl) imide (LiFSI)

| Base composition | Additive | FIG. |
|---|---|---|
| 95% 1M LiFSI in Propylene | 5% 2-(2,2,2-trifluoroethyl)- | 4a |
| carbonate | 1,3-Dioxolane | |
| 85% 1M LiFSI in Propylene | 15% 2-(2,2,2-trifluoroethyl)- | 4b |
| carbonate | 1,3-Dioxolane | |
| 25% 1M LiFSI in Propylene | 75% 2-(2,2,2-trifluoroethyl)- | 4c |
| carbonate | 1,3-Dioxolane | |
| 95% 1M LiFSI in a mixture of | 5% 2-(2,2,2-trifluoroethyl)- | 5a |
| Propylene carbonate (90%) and | 1,3-Dioxolane | |
| fluoroethylene carbonate (10%) | | |
| 85% 1M LiFSI in a mixture of | 15% 2-(2,2,2-trifluoroethyl)- | 5b |
| Propylene carbonate (90%) and | 1,3-Dioxolane | |
| fluoroethylene carbonate (10%) | | |
| 25% 1M LiFSI in a mixture of | 75% 2-(2,2,2-trifluoroethyl)- | 5c |
| Propylene carbonate (90%) and | 1,3-Dioxolane | |
| fluoroethylene carbonate (10%) | | |
| 95% 1M LiFSI in a mixture of | 5% 2-(2,2,2-trifluoroethyl)- | 6a |
| ethylene carbonate (30%) and | 1,3-Dioxolane | |
| ethyl methyl carbonate (70%) | | |
| 85% 1M LiFSI in a mixture of | 15% 2-(2,2,2-trifluoroethyl)- | 6b |
| ethylene carbonate (30%) and | 1,3-Dioxolane | |
| ethyl methyl carbonate (70%) | | |
| 25% 1M LiFSI in a mixture of | 75% 2-(2,2,2-trifluoroethyl)- | 6c |
| ethylene carbonate (30%) and | 1,3-Dioxolane | |
| ethyl methyl carbonate (70%) | | |

### FIGURES

FIGS. 1a to 1c show ¹⁹F NMR spectra of LiPF6 and 2-(2,2,2-trifluoroethyl)-1,3-Dioxolane in propylene carbonate.

FIGS. 2a to 2c show ¹⁹F NMR spectra of LiPF6 and 2-(2,2,2-trifluoroethyl)-1,3-Dioxolane in propylene carbonate (90%) and fluoroethylene carbonate (10%).

FIGS. 3a to 3c show ¹⁹F NMR spectra of LiPF6 and 2-(2,2,2-trifluoroethyl)-1,3-Dioxolane in ethylene carbonate (30%) and ethyl methyl carbonate (70%).

FIGS. 4a to 4c show ¹⁹F NMR spectra of LiFSI and 2-(2,2,2-trifluoroethyl)-1,3-Dioxolane in propylene carbonate.

FIGS. 5a to 5c show ¹⁹F NMR spectra of LiFSI and 2-(2,2,2-trifluoroethyl)-1,3-Dioxolane in propylene carbonate (90%) and fluoroethylene carbonate (10%).

FIGS. 6a to 6c show ¹F NMR spectra of LiFSI and 2-(2,2,2-trifluoroethyl)-1,3-Dioxolane in ethylene carbonate (30%) and ethyl methyl carbonate (70%).

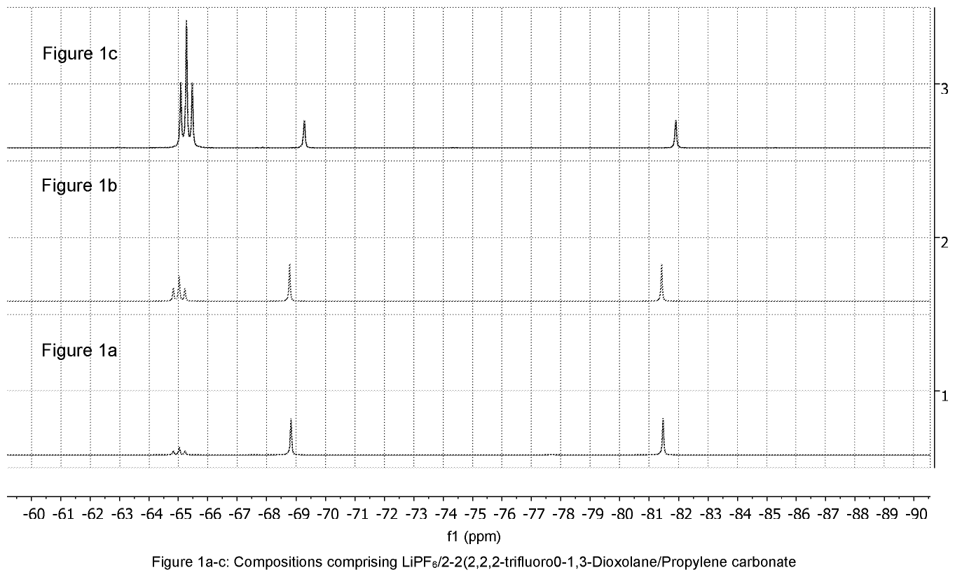

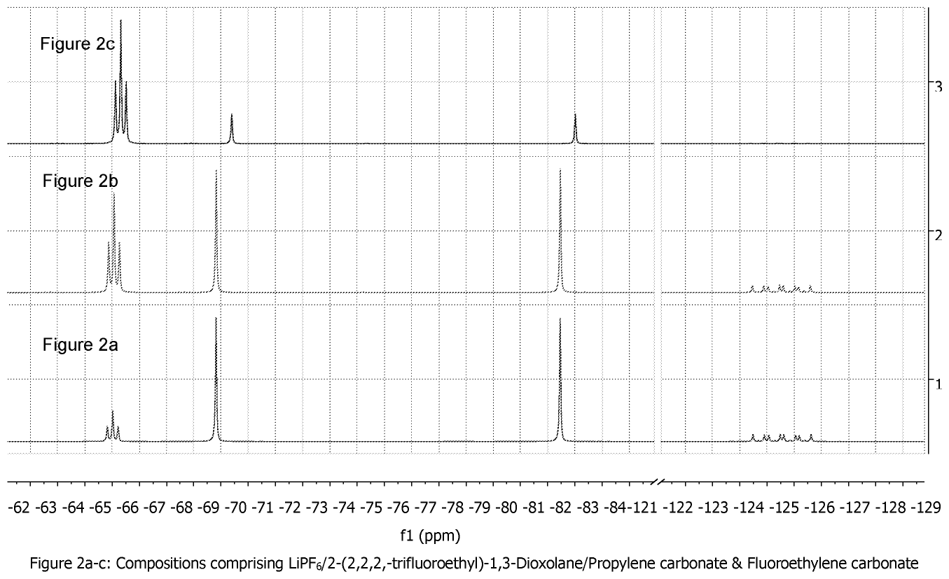

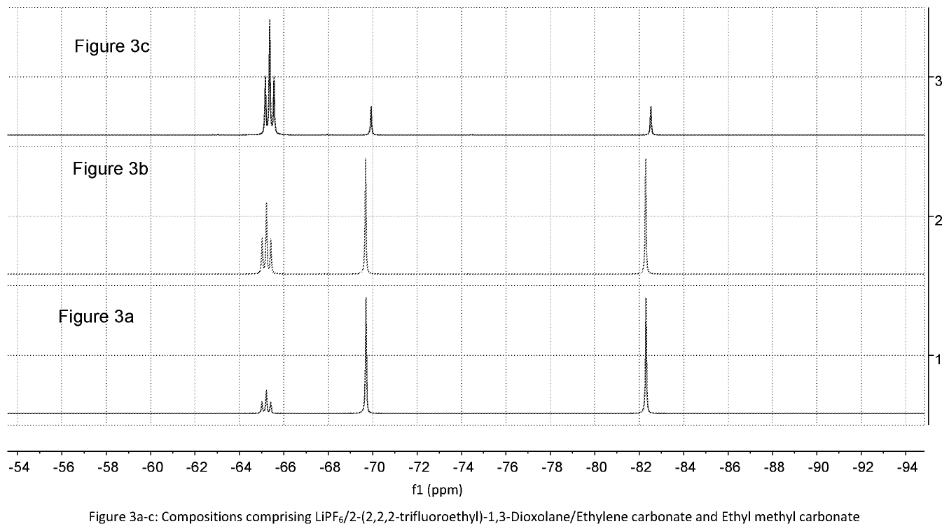

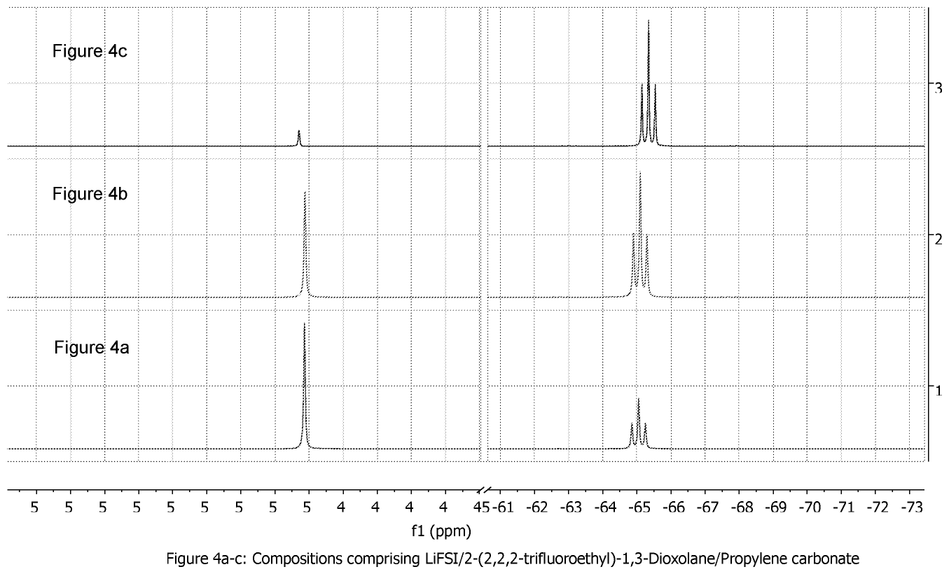

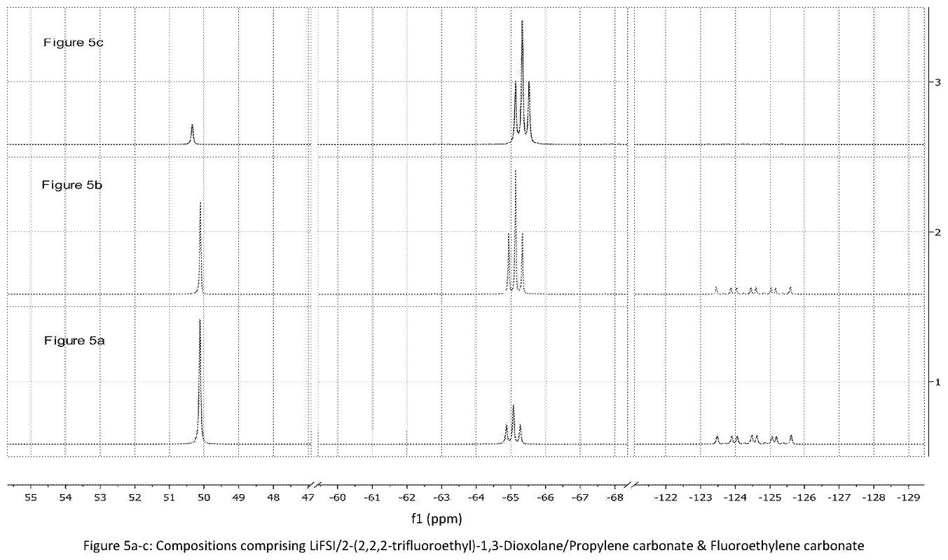

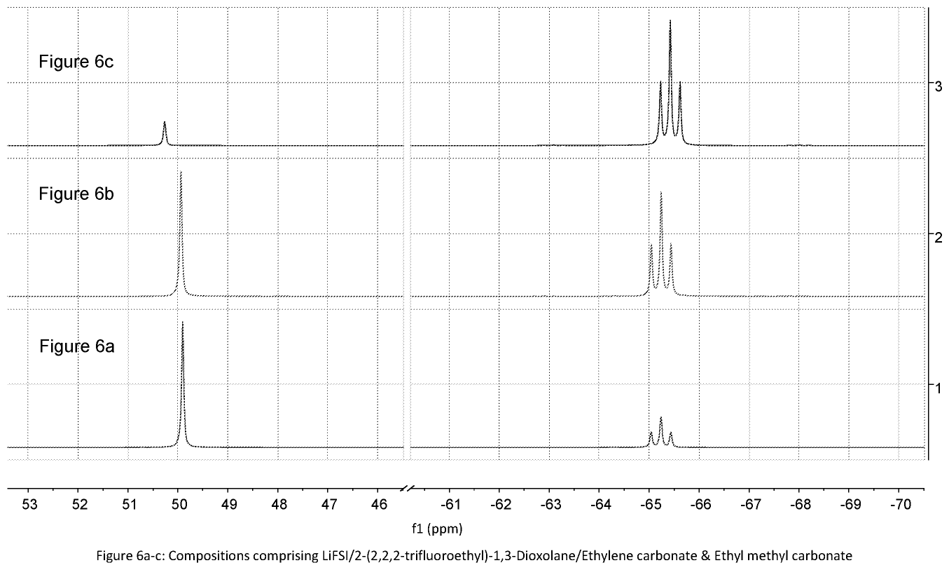

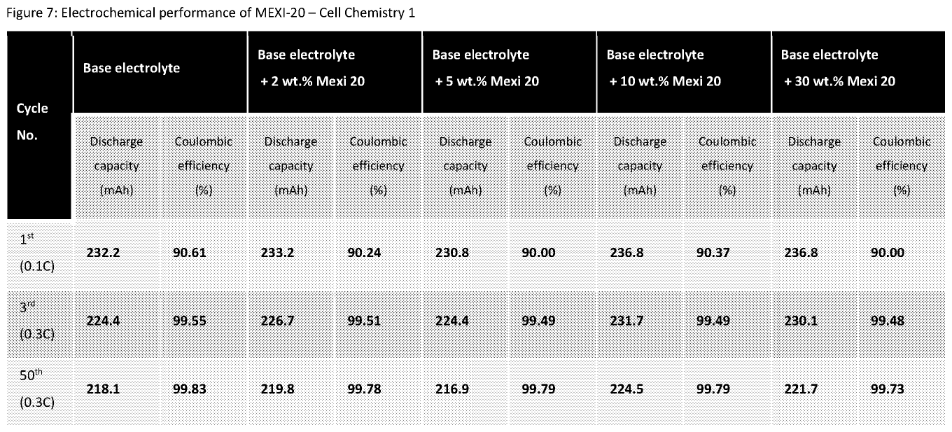

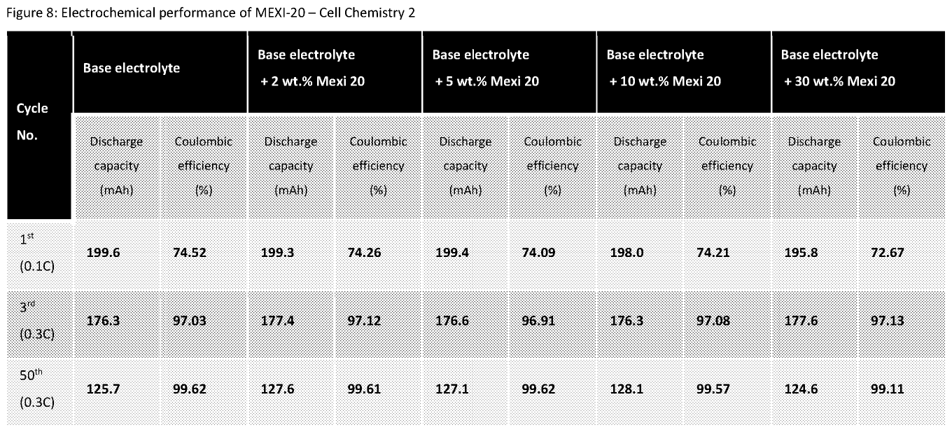

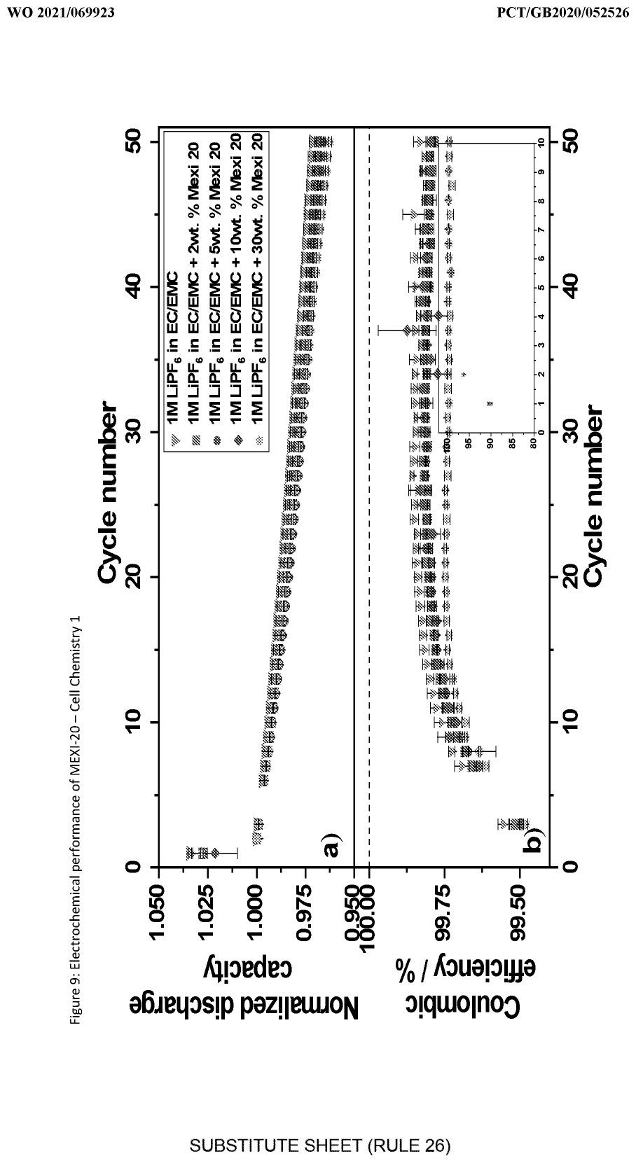

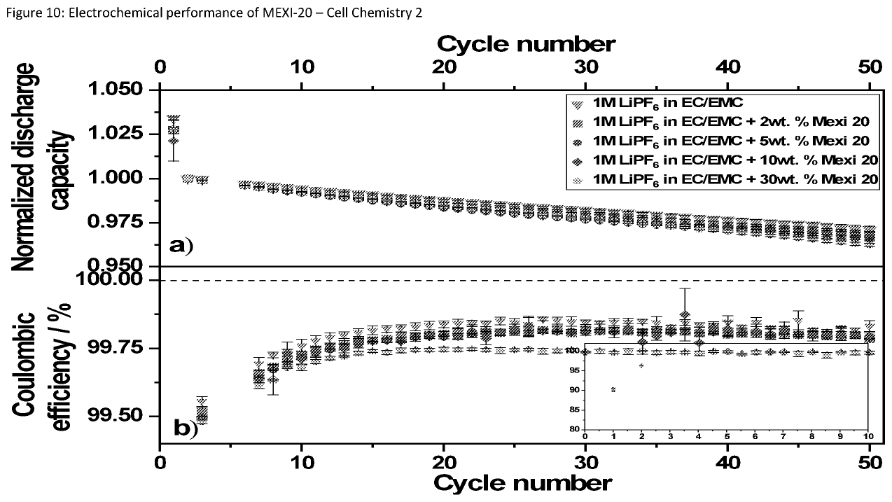

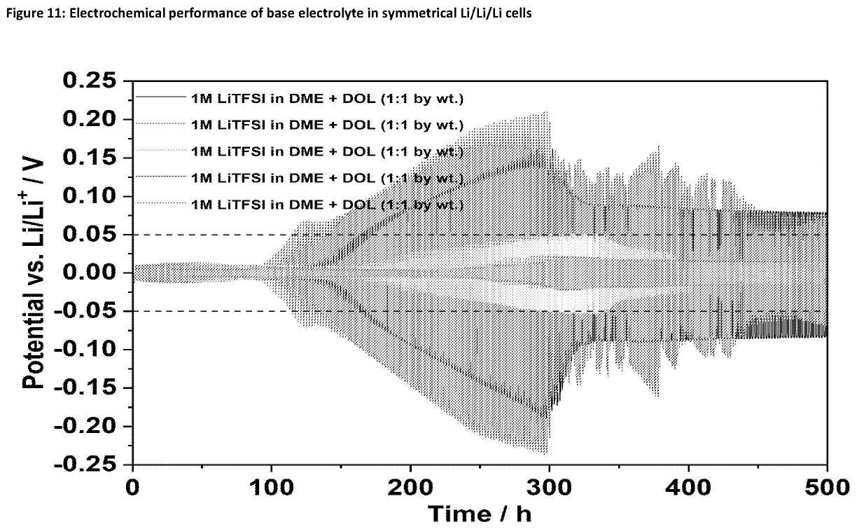

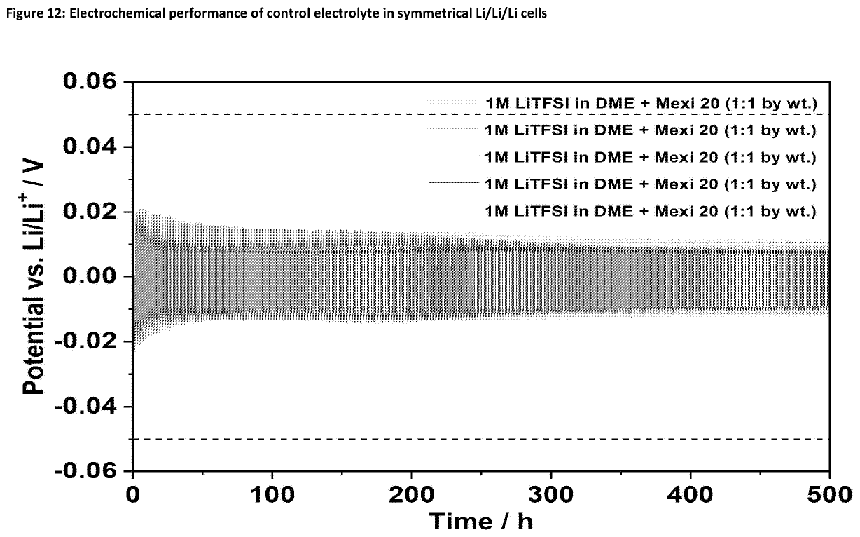

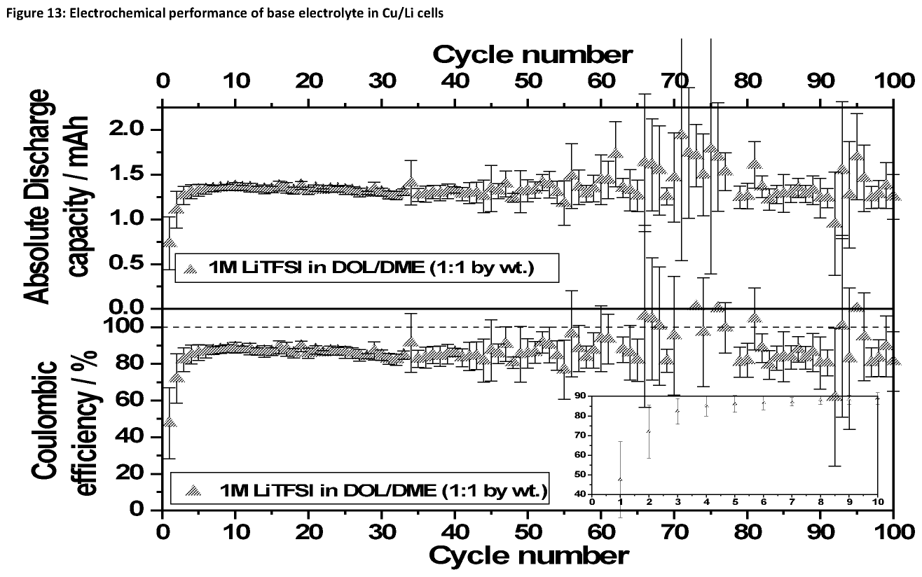

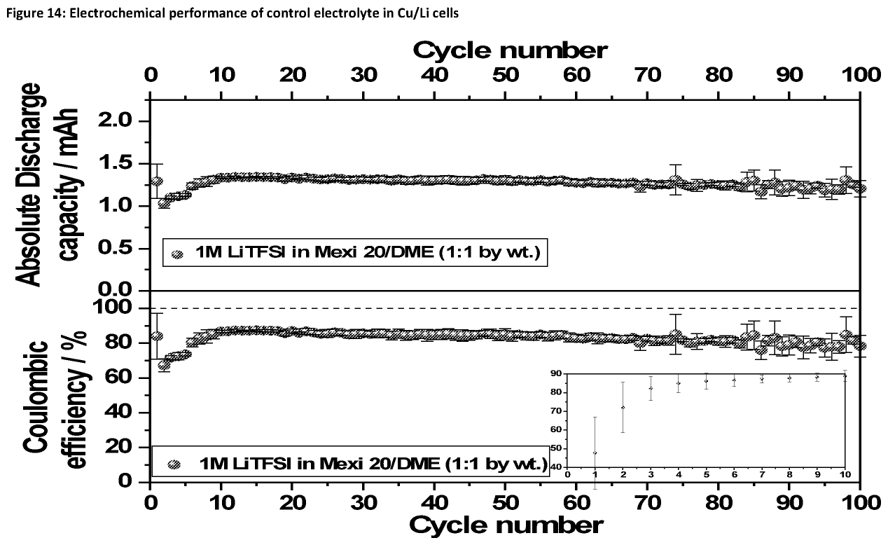

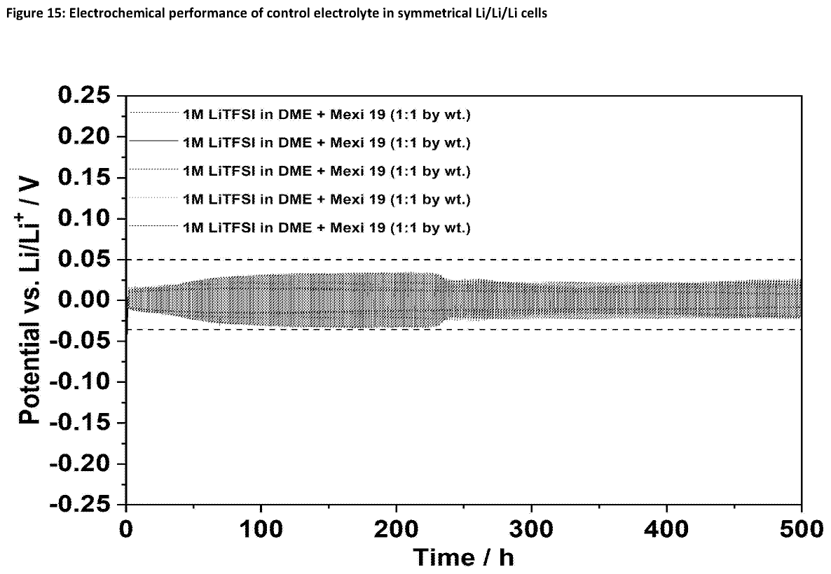

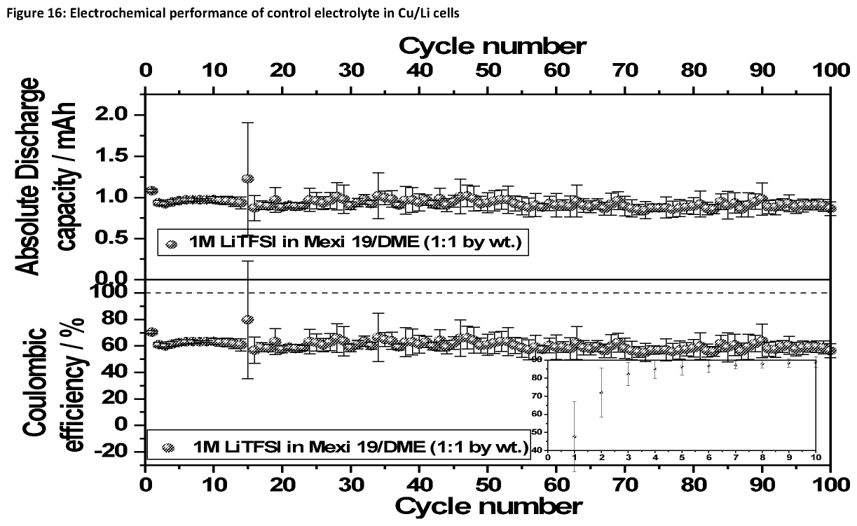

### FLAMMABILITY AND SAFETY TESTING

For convenience 2-(2,2,2-trifluoroethyl)-1,3-Dioxolane will be referred to as Mexi-20 hereafter.

Flash Point

Flashpoints were determined using a Miniflash FLP/H device from Grabner Instruments following the ASTM D6450 standard method:

| | Concentration (% wt) in standard electrolyte | | | | | |
|---|---|---|---|---|---|---|
| | 1M LiPF₆ in (30% ethylene carbonate & | | | | | |
| | 70% of ethyl methyl carbonate | | | | | |
| | 0 | 2 | 5 | 10 | 30 | 100 |
| Component | Flashpoint (° C.) | | | | | |
| 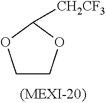 | 32 ± 2 | 36 ± 2 | 32 ± 2 | 39 ± 4 | 37 ± 1 | 41 ± 1 |

These measurements demonstrate that the addition of the additive designated MEXI-20 raised the flashpoint of the standard electrolyte solution.

Self-Extinguishing Time

Self-extinguishing time was measured with a custom-built device that contained an automatically controlled stopwatch connected to an ultraviolet light detector. In this experiment, the electrolyte to be examined (500 μL) was applied to a Whatman GF/D (0=24 mm) glass microfiber filter. An ignition source was transferred under the sample and held in this its position for a preset time (1, 5 or 10 seconds) to ignite the sample. Ignition and burning of the sample were detected using a UV light detector. Evaluation is carried out by plotting the burning time/weight of electrolyte [s·g⁻¹] over ignition time [s], and extrapolation by linear regression line to ignition time=0 s. The self-extinguishing time (s·g⁻¹) is the time that is needed until the sample stops burning once inflamed.

| | Concentration (% wt) in standard electrolyte | | | | | |
|---|---|---|---|---|---|---|
| | 1M LiPF₆ in (30% ethylene carbonate & | | | | | |
| | 70% ethyl methyl carbonate | | | | | |
| | 0 | 2 | 5 | 10 | 30 | 100 |
| Component | Self-extinguishing time (s.g⁻¹) | | | | | |
| 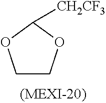 | 39 ± 2 | 28 ± 4 | 27 ± 2 | 27 ± 2 | 29 ± 2 | 28 ± 2 |

These measurements demonstrate that the compound MEXI-20 has flame retarding properties.

Electrochemical Testing

Lithium-Ion Batteries

Drying

Before testing, MEXI-20 was dried to less than 10 ppm water by treatment with a pre-activated Type 4A molecular sieve.

Electrolyte Formulation

Electrolyte preparation and storage was carried out in an Argon-filled glove box (H₂O and O₂<0.1 ppm). The base electrolyte was 1M LiPF₆ in ethylene carbonate:ethyl methyl carbonate (3:7 wt. %) with MEXI-20 additive at concentrations of 2, 5, 10 and 30 wt. %.

Cell Chemistry and Construction

The performance of each electrolyte formulation was tested in multi-layer pouch cells over 50 cycles (2 cells per electrolyte):
Chemistry 1: Lithium-Nickel-Cobalt-Manganese-Oxide (NCM622) positive electrode and artificial graphite (specific capacity: 350 mAh g⁻¹) negative electrode. The area capacity of NMC622 and graphite amounted to 3.5 mAh cm⁻² and 4.0 mAh cm⁻², respectively. The N/P ratio amounted to 115%.
Chemistry 2: Lithium-Nickel-Cobalt-Manganese-Oxide (NCM622) positive electrode and SiOₓ/graphite (specific capacity: 550 mAh g⁻¹) negative electrode. The area capacity of NMC622 and SiOₓ/graphite amount to 3.5 mAh/cm⁻² and 4.0 mAh cm⁻², respectively. The N/P ratio amounted to 115%.

The test pouch cells had the following characteristics:
Nominal capacity 240 mAh+/−2%
Standard deviations:
   Capacity: ±0.6 mAh
   Coulombic Efficiency (CE) 1^{st} cycle: ±0.13%
   Coulombic Efficiency (CE) subsequent cycles: ±0.1%
Positive electrode: NMC-622
   Active material content: 96.4%
   Mass loading: 16.7 mg cm⁻²
Negative electrode: Artificial Graphite
   Active material content: 94.8%
   Mass loading: 10 mg cm⁻²
   Separator PE (16 μm)+4 μm Al₂O₃
   Balanced at cut-off voltage of 4.2 V
Negative electrode: Artificial graphite+SiO
   Active material content: 94.6%
   Mass loading: 6.28 mg cm⁻²
   Separator: PE (16 μm)+4 μm Al₂O₃
   Balanced at cut-off voltage of 4.2 V

After assembly the following formation protocol was used (CC=Constant Current charging and CCCV=Constant Current Constant Voltage charging):
1. Step charge to 1.5 V followed by 5 h rest step (wetting step @40° C.)
2. CCCV (C/10, 3.7 V (Iₗᵢₘᵢₜ: 1 h)) (preformation step)
3. Rest step (6 h)
4. CCCV (C/10, 4.2 V (Iₗᵢₘᵢₜ: 0.05 C)) rest step (20 min)
5. CC discharge (C/10, 3.8 V), (degassing of the cell)
6. CC discharge (C/10, 2.8 V)

Following this formation step, the cells were tested as follows:
Rest step (1.5 V, 5 h), CCCV (C/10, 3.7 V (1 h))
Rest step (6 h), CCCV (C/10, 4.2 V (Iₗᵢₘᵢₜ: 0.05 C))
Rest step (20 min), CC discharge (C/10, 3.8 V)
Degassing step
Discharge (C/10, 2.8 V), rest step (5 h)
CCCV (C/3, 4.2 V (Iₗᵢₘᵢₜ: 0.05 C)), rest step (20 min)
CC discharge (C/3, 2.8 V)
50 cycles or until 50% SOH is reached at 40° C.:
CCCV (C/3, 4.2 V (Iₗᵢₘᵢₜ: 0.02 C)), rest step (20 min)
CC discharge (C/3, 3.0 V), rest step (20 min)

Test Results

The test results for the additive MEXI-20 in each cell chemistry are summarised in FIGS. 7-10.

Lithium Metal Batteries

Symmetrical Li/Li/Li Cells

Electrolyte Formulation

The electrolyte solutions were prepared and stored in an Argon filled glove box (H₂O and O₂<0.1 ppm):
Base electrolyte: 1M LiTFSI in Dimethoxyethane (DME):Dioxolane (DOL) (1:1 wt. %)
Control electrolyte: 1M LiTFSI in DME:Mexi-20 (1:1 wt. %)

Cell Construction, Testing and Chemistry

Symmetrical 3 electrode Li/electrolyte/Li cells (“Swagelok cells”) were filled with base and control electrolyte and used for electrochemical testing and for the determination of key performance indicators thereof (5 cells per electrolyte, in total 10 cells).

This cell chemistry was chosen because it is regarded as the “state of the art” cell chemistry for measuring the stripping and plating behavior of metallic lithium, as well as the evolution of overpotential during stripping and plating. The symmetrical Li/Li/Li cells were cycled at 0.1 mA/cm². Charge and discharge times were 1 h each (a cycle is defined as a charging step followed by a discharge step). The cells were cycled for 25 days at 20° C.

Results

The results are shown in FIGS. 11 and 12.

For the base electrolyte, an exponential increase of overpotentials during continuous stripping and plating of lithium metal was observed. Additionally, a strong increase of the cell resistance was observed and was caused by a large degradation of the electrolyte on the Li electrode. For the control electrolyte, constant and low overpotentials were observed during continuous stripping and plating of lithium metal. The overpotentials remained stable over the whole course of the measurement. The investigated compound (MEXI-20) shows the ability to form or modify the SEI on lithium in concentrations 50 wt. % and in combination with DME as co-solvent.

These electrochemical test results for Li/Li/Li cells show that the cycling performance of the cells was positively influenced by MEXI-20 in concentrations of 50 wt. %. Electrolytes containing MEXI-20 displayed constant and comparable low overpotentials during continuous stripping and plating of lithium metal. This is thought to be because the MEXI-20-containing electrolyte formed a less resistive passivation layer on lithium metal, therefore MEXI-20 can be beneficially used as co-solvent in rechargeable lithium metal batteries.

Cu/Li Cells

Electrolyte Formulation

The electrolyte preparation and storage were carried out in an argon filled glove box (H₂O and O₂<0.1 ppm).
Base electrolyte: 1M LiTFSI in DME:DOL (1:1 wt. %)
Control electrolyte: 1M LiTFSI in DME:Mexi-20 (1:1 wt. %)

Cell Construction, Testing and Chemistry

Two electrode Cu/electrolyte/Li cells (coin cells) filled with base and control electrolyte were used for electrochemical testing and for the determination of key performance indicators (5 cells per electrolyte, in total 10 cells).

This cell chemistry was chosen because it represents the “state of the art” cell chemistry for measuring the stripping and plating behavior of metallic lithium as well as the evolution of the Coulombic efficiency during cycling.

Cycling: The cells were cycled at 1 mA/cm² for 1 h for the charge process (lithium deposition) and 0.25 mA/cm² for the discharge process (lithium dissolution) until the cut-off voltage for the Cu electrode of 1 V was reached (a cycle is defined as a charge step followed by a discharge step). Cells were cycled for 100 cycles (˜25 days) at 20° C.

Results

The test results are summarised in FIGS. 13-14.

FIG. 13 illustrates the test data for the base electrolyte: 1M LiTFSI in DME:DOL (1:1 wt. %):
There was an increase in the Coulombic efficiency of the cells (CE) during the first 10 cycles up to ˜88%
There was increased fluctuation in the discharge capacity and CE during continuous stripping and plating of lithium metal after 35 cycles
This strong increase is thought to be caused by a continuous degradation of the electrolyte on the Cu electrode and the formation of high surface area lithium metal
The increase in the CE beyond 100% is a clear indication for dendrite formation and growth causing small short circuits in the cell and leading to cell performance degradation

FIG. 14 illustrates the test data for the control electrolyte: 1M LITFSI in DME:Mexi 20 (1:1 wt %):
There was an increase in the CE of the cells within the first 10 cycles up to ˜88%
The discharge capacity and CE evolution was stable during continuous stripping and plating of lithium metal
The CE and discharge capacity showed only limited fading over 100 cycles
This stable performance is an indication for the formation of a more effective surface layer in presence of Mexi-20 in the electrolyte
There was no evidence for the formation of dendrites and cell short circuits

These results show that the substitution of DOL with Mexi-20 leads to more stable cycling performance with fewer parasitic reactions. There was no evidence for the formation of lithium metal dendrites and short circuits in the cells containing Mexi-20 and the of Mexi-20 containing electrolyte led to a strong increase in the 1′ cycle CE.

These results further confirm the utility of Mexi-20 In secondary (rechargeable) batteries with lithium metal anodes.

### Example 2

### Synthesis and Isolation of 5-Trifluoromethyl-2-(2,2,2-Trifluoroethyl)-1,3-Dioxolane (Mexi-19)

Mexi-19 was prepared by the cycloaddition of 3,3,3-Trifluoropropanal and 3,3,3-Trifluoropropane-1,2-diol:

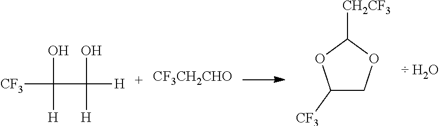

3,3,3-Trifluoropropanal was freshly prepared from 3,3,3-Trifluoropropionaldehyde hydrate by dehydrating it with phosphorous pentoxide and used immediately. 850 g of the freshly prepared aldehyde was charged to a 5-liter flask equipped with a magnetic stirrer, heating mantle, modified Dean Stark trap, and reflux condenser. 930 grams (7.15 moles) of 3,3,3-Trifluoropropane-1,2-diol was added, followed by two liters of Methylene Chloride and 20 grams of p-Toluenesulfonic Acid Monohydrate catalyst. The mixture was refluxed vigorously for 48 hours whilst the water generated by the reaction was continuously removed.

After cooling to room temperature, the reaction mixture was neutralized by washing with one liter of saturated aqueous sodium bicarbonate solution in a separatory funnel followed by two washes with one liter of water.

The organic (bottom) phase was dried with anhydrous magnesium sulfate and filtered. Dichloromethane and unreacted aldehyde were stripped off on a rotary evaporator. The crude product was fractionally distilled using a spinning band column to give a product of >99.6% purity (GC area %), boiling point 58° to 60° C. @ 25 mmHg. Two diastereomers were present in a ratio of approximately 5:1. Yield 1,046 grams (65% of theoretical based on limiting diol reactant).

The product was dried over 3 Å molecular sieves and its structure confirmed by ¹⁹F NMR spectroscopy: ¹⁹F NMR (56 MHz) δ −61.4, 61.7 (t, J=10.6 Hz), 75.6, 76.7 (d, J=6.2 Hz).

The flash point of Mexi-19 was determined to be 138±4° C. as described above.

The electrochemical performance of Mexi-19 in symmetrical Li/Li/Li cells and Cu/Li was determined in the same way as described above for Mexi-20 having first dried it by treatment with a 4A molecular sieve.

Electrochemical Test Results in Symmetrical Li/Li/Li Cells

The test results are illustrated in FIG. 15 (and should be compared with FIG. 11). As was the case for Mexi-20, the cells constructed using Mexi-19 containing electrolyte displayed constant and comparable low overpotentials during continuous stripping and plating of lithium metal. Furthermore, the overpotentials remained stable over the whole course of the measurements. This behavior was found to be highly reproducible in several repetitions of the experiment.

Electrochemical Test Results in Symmetrical Cu/Li Cells

The test results are illustrated in FIG. 16 (and should be compared with FIG. 13). Whilst Mexi-19 showed a lower CE for the lithium stripping and plating from and onto the Cu electrode compared to the base electrolyte it was clear that the discharge capacity and CE were more stable. Furthermore, there was very little fading of the CE and discharge capacity over 100 cycles. This stable performance is indicative of the formation of a more effective surface layer on Cu in presence of Mexi-19. There was no evidence for the formation of lithium metal dendrites and short circuits in the cell during cycling.

1. A nonaqueous battery electrolyte formulation, comprising a compound of Formula 1: 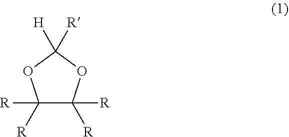
wherein R′ and each R are, independently, H, F, Cl, CF₃, alkyl, or fluoroalkyl.

2. The formulation according to claim 1, wherein the alkyl and the fluoroalkyl have a carbon chain length of from C₁ to C₆.

3. The formulation according to claim 1, further comprising a metal electrolyte salt, present in an amount of from 0.1 to 20 wt % relative to a total mass of the formulation, wherein the metal salt is a salt of lithium, sodium, magnesium, calcium, lead, zinc, or nickel.

4. (canceled)

5. The formulation according to claim 3, wherein the metal salt is a salt of lithium selected from the group consisting of lithium hexafluorophosphate (LiPF₆), lithium tetrafluoroborate (LiBF₄), lithium perchlorate (LiClO₄), lithium triflate (LiSO₂CF₃), lithium bis(fluorosulfonyl)imide (LiFSI, Li(FSO₂)₂N), and lithium bis(trifluoromethanesulfonyl)imide (LiTFSI, Li(CF₃SO₂)₂N).

6. The formulation according to claim 1, further comprising an additional solvent in an amount of from 0.1 wt % to 99.9 wt % of a liquid component of the formulation, wherein the additional solvent is selected from the group consisting of fluoroethylene carbonate (FEC), propylene carbonate (PC), ethylene carbonate (EC), dimethoxyethane, and thionyl chloride.

7-9. (canceled)

10. The formulation according to claim 1, further comprising a metal ion, optionally in combination with a solvent.

11. A battery comprising the formulation according to claim 1.

12. The battery according to claim 11, further comprising a negative electrode that is lithium metal,
wherein the formulation comprises dimethoxyethane, and lithium bis(fluorosulfonyl)imide and/or lithium bis(trifluoromethanesulfonyl)imide, and
wherein the battery is a secondary battery.

13-16. (canceled)

17. A method of reducing the flammability of a battery and/or a battery electrolyte, the method comprising adding to the battery and/or the battery electrolyte the formulation according to claim 1.

18. A method of powering an article comprising a battery, the method comprising adding to the battery a battery electrolyte formulation comprising a compound of Formula 1: 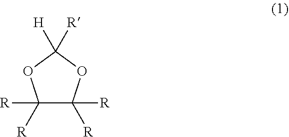
wherein R′ and each R are, independently, H, F, Cl, CF₃, alkyl, or fluoroalkyl.

19. A method of retrofitting a battery electrolyte, the method comprising (a) at least partially replacing the battery electrolyte with the formulation according to claim 1; and/or (b) supplementing the battery electrolyte with the formulation.

20. A method of preparing the formulation according to claim 5, the method comprising mixing a compound of Formula 1 with ethylene, propylene, or fluoroethylene carbonate and with the salt of lithium so as to produce the formulation.

21. The method according to claim 18, wherein a capacity of the battery and/or a charge transfer within the battery is improved relative to a battery without the formulation.

22. The method according to claim 18, wherein the formulation comprises a metal electrolyte salt, present in an amount of from 0.1 to 20 wt % relative to a total mass of the formulation, wherein the metal salt is a salt of lithium, sodium, magnesium, calcium, lead, zinc, or nickel.

23. (canceled)

24. The method according to claim 18, wherein the metal salt is a salt of lithium selected from the group consisting of lithium hexafluorophosphate (LiPF₆), lithium perchlorate (LiClO₄), lithium tetrafluoroborate (LiBF₄), lithium triflate (LiSO₂CF₃), lithium bis(fluorosulfonyl)imide (LiFSI, Li(FSO₂)₂N), and lithium bis(trifluoromethanesulfonyl)imide (LiTFSI, Li(CF₃SO₂)₂N).

25. The method according to claim 18, wherein the formulation comprises an additional solvent in an amount of from 0.1 wt % to 99.9 wt % of a liquid component of the formulation, and wherein the additional solvent is selected from the group consisting of fluoroethylene carbonate (FEC), propylene carbonate (PC), ethylene carbonate (EC), dimethoxyethane, and thionyl chloride.

26. (canceled)

27. A method of preparing the compound according to claim 1, the method comprising (a) condensing a glycol or a diol with an aldehyde, or (b) reacting a glycol with trifluoromethyl acetylene at a positive pressure under basic conditions, so as to produce the compound.

28. The method according to claim 27 wherein the glycol is a compound of Formula 2:

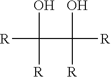

29. The method according to claim 27, wherein the method is carried out at a temperature above 0° C.

30. The method according to claim 27, wherein the method is carried out for from 9 to 10 hours.

31. The method according to claim 27, wherein the positive pressure is maintained at from 8 to 12 barg.

32. The method according to claim 27, wherein the compound is:

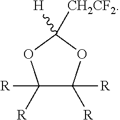

33. (canceled)

34. The method according to claim 27, in wherein the aldehyde is a compound of Formula 3:

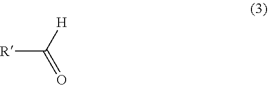

35. The method according to claim 27, wherein the method is carried out in the presence of an acid catalyst.
